(19) United States
(12) Patent Application Publication
(10) Pub. No.: US 2025/0154594 A1
(43) Pub. Date: May 15, 2025

(54) BLOOD BIOMARKER FOR DIAGNOSIS, PROGNOSIS PREDICTION, OR TREATMENT OF PARKINSON’S DISEASE, METHOD FOR DIAGNOSING PARKINSON’S DISEASE, OR PREDICTING PROGNOSIS USING THE SAME, AND METHOD FOR SCREENING SUBSTANCES FOR TREATING PARKINSON’S DISEASE

(71) Applicant: THE CATHOLIC UNIVERSITY OF KOREA INDUSTRY-ACADEMIC COOPERATION FOUNDATION, Seoul (KR)

(72) Inventors: Ko Eun CHOI, Seoul (KR); Joong-Seok KIM, Seoul (KR)

(73) Assignee: THE CATHOLIC UNIVERSITY OF KOREA INDUSTRY-ACADEMIC COOPERATION FOUNDATION, Seoul (KR)

(21) Appl. No.: 18/941,550

(22) Filed: Nov. 8, 2024

(30) Foreign Application Priority Data
Nov. 9, 2023 (KR) 10-2023-0154873
Jan. 31, 2024 (KR) 10-2024-0015332
Nov. 1, 2024 (KR) 10-2024-0153730

(51) Int. Cl. C12Q 1/6883; C12Q 1/6874

(52) U.S. Cl. CPC C12Q 1/6883; C12Q 1/6874; C12Q 2600/118; C12Q 2600/178

(57) ABSTRACT

Provided are a blood biomarker for diagnosis, prognosis prediction, or treatment of Parkinson's disease (PD), a method for diagnosing or predicting the prognosis of PD using the same, and a method for screening substances for treating PD, where it is confirmed that the levels of 14 types of miRNA are significantly increased or decreased in the blood of PD patients, and the 14 types of miRNA reflect the correlation between genes and environmental factors identified in subjects with PD, thus the 14 types of miRNA are expected to be used as a diagnostic and prognostic biomarker as well as a new therapeutic target related to the pathological mechanism of PD.

### BACKGROUND

### Field of the Invention

The present invention relates to a blood biomarker for diagnosis, prognosis prediction, or treatment of Parkinson's disease (PD), a method for diagnosing PD, or predicting a prognosis using the same, and a method for screening substances for treating PD.

### Discussion of Related Art

PD is a degenerative disease of the central nervous system whose pathophysiological symptoms include deformation of the substantia nigra pars compacta of the midbrain, reduction in brain volume, and aggregation of α-synuclein (aSyn) accompanied by bradykinesia, resting tremor, rigidity and gait disturbance. Abnormal accumulation of phosphorylated aSyn throughout the brain and dopamine deficiency due to neuronal loss in the substantia nigra (SN) are diagnostic markers of PD pathology.

Recent advances in genomic technology have led to the successful identification of a large number of genes that play a role in PD pathogenesis associated with several biological pathways, including aSyn proteolysis, mitochondrial function, oxidative stress, calcium homeostasis, axonal transport, and neuroinflammation. In particular, it has been reported that frequent point mutations appear in SNCA (PARK1/4), PRKN (PARK2), PINK1 (PARK6), LRRK2 (PARK8), and GBA genes in the case of familial PD.

However, although several large-scale genome-wide association studies have been conducted to discover genes or diagnostic markers associated with sporadic forms of PD, which account for 90% to 95% of all PD patients, no marker that can predict the risk of PD or make a diagnosis with high accuracy has been developed to date.

Meanwhile, the number of PD patients in Korea was 61,565 in 2010, and the number increased to 85,888 in 2014 at an average annual increase rate of 8.7%. In 2014, the proportion of patients aged 60 or older was 95.7%, and the prevalence rate exhibited a correlation with the patient's age. With regard to the proportions according to sex, the patients included 33,831 men and 52,057 women.

Compared to Alzheimer's disease, the onset of PD is much earlier, which is at around 60 years of age. As a result, the progression of the disease continues for 20 years or longer, so there is an urgent need for a treatment method that may slow down the progression of the disease through early detection.

### RELATED ART DOCUMENT

### Patent Document

Korean Patent Publication No. 10-2018-0098153

### SUMMARY OF THE INVENTION

### Technical Problem

An object of the present invention is to provide a method comprising the steps of:
(S1) measuring the level of any one or more miRNAs selected from the group consisting of hsa-miR-11401, has-miR-12136, hsa-miR-145-3p, hsa-miR-2115-3p, hsa-miR-23a-3p, hsa-miR-27a-3p, hsa-miR-28-3p, hsa-miR-331-3p, hsa-miR-4435, hsa-miR-4507, hsa-miR-4685-3p, hsa-miR-501-3p, hsa-miR-618, and hsa-miR-7109-3p in a biological sample isolated from a subject;
(S2) comparing the level of the miRNA with the level of the miRNA in a biological sample isolated from a normal control group;
(S3) diagnosing a subject as having Parkinson's disease (PD), when among the miRNAs in a biological sample isolated from the subject:
(a) the level of one or more miRNAs selected from the group consisting of hsa-miR-11401, hsa-miR-28-3p, hsa-miR-331-3p, hsa-miR-4435, hsa-miR-4507, hsa-miR-4685-3p, hsa-miR-501-3p, and hsa-miR-7109-3p is increased compared to the level of the miRNAs in a biological sample isolated from a normal control group; or
(b) the level of one or more miRNAs selected from the group consisting of hsa-miR-12136, hsa-miR-145-3p, hsa-miR-2115-3p, hsa-miR-23a-3p, hsa-miR-27a-3p, and hsa-miR-618 is decreased compared to the level of the miRNAs in a biological sample isolated from a normal control group; and
(S4) treating the PD with chemotherapy, surgery, or biological therapy when PD is diagnosed in step (S3).

Another object of the present invention is to provide a method comprising the steps of:
(S1) measuring the level of any one or more miRNAs selected from the group consisting of hsa-miR-11401, has-miR-12136, hsa-miR-145-3p, hsa-miR-2115-3p, hsa-miR-23a-3p, hsa-miR-27a-3p, hsa-miR-28-3p, hsa-miR-331-3p, hsa-miR-4435, hsa-miR-4507, hsa-miR-4685-3p, hsa-miR-501-3p, hsa-miR-618, and hsa-miR-7109-3p in a biological sample isolated from a subject;
(S2) comparing the level of the miRNA with the level of the miRNA in a biological sample isolated from a normal control group;
(S3) diagnosing a subject as having PD, when among the miRNAs in a biological sample isolated from the subject:
(a) the level of one or more miRNAs selected from the group consisting of hsa-miR-11401, hsa-miR-28-3p, hsa-miR-331-3p, hsa-miR-4435, hsa-miR-4685-3p, and hsa-miR-501-3p is more than 2 fold change increase compared to the level of the miRNAs in a biological sample isolated from a normal control group, and the AUC value is ≥0.5, the p-value is <0.5, the Confidence Limits value falls between 0.001 and 165; or
(b) the level of one or more miRNAs selected from the group consisting of hsa-miR-145-3p, hsa-miR-23a-3p, hsa-miR-27a-3p, and hsa-miR-618 is more than 2 fold change decreased compared to the level of the miRNAs in a biological sample isolated from a normal control group, and the AUC value is >0.5, the p-value is <0.5, the Confidence Limits value falls between 0.001 and 165; and
(S4) treating the PD with chemotherapy, surgery, or biological therapy when PD is diagnosed in step (S3).

However, the technical problems to be solved by the present invention are not limited to the problems mentioned above, and other problems that are not mentioned can be clearly understood from the description below by those skilled in the art to which the present invention pertains.

### Technical Solution

The present invention provides a method comprising the steps of:
(S1) measuring the level of any one or more miRNAs selected from the group consisting of hsa-miR-11401, has-miR-12136, hsa-miR-145-3p, hsa-miR-2115-3p, hsa-miR-23a-3p, hsa-miR-27a-3p, hsa-miR-28-3p, hsa-miR-331-3p, hsa-miR-4435, hsa-miR-4507, hsa-miR-4685-3p, hsa-miR-501-3p, hsa-miR-618, and hsa-miR-7109-3p in a biological sample isolated from a subject;
(S2) comparing the level of the miRNA with the level of the miRNA in a biological sample isolated from a normal control group;
(S3) diagnosing a subject as having Parkinson's disease (PD), when among the miRNAs in a biological sample isolated from the subject:
(a) the level of one or more miRNAs selected from the group consisting of hsa-miR-11401, hsa-miR-28-3p, hsa-miR-331-3p, hsa-miR-4435, hsa-miR-4507, hsa-miR-4685-3p, hsa-miR-501-3p, and hsa-miR-7109-3p is increased compared to the level of the miRNAs in a biological sample isolated from a normal control group; or
(b) the level of one or more miRNAs selected from the group consisting of hsa-miR-12136, hsa-miR-145-3p, hsa-miR-2115-3p, hsa-miR-23a-3p, hsa-miR-27a-3p, and hsa-miR-618 is decreased compared to the level of the miRNAs in a biological sample isolated from a normal control group; and
(S4) treating the PD with chemotherapy, surgery, or biological therapy when PD is diagnosed in step (S3), but not limited thereto.

In one embodiment of the present invention, the biological sample may be any one selected from the group consisting of blood, serum, whole blood, and plasma, but is not limited thereto.

The present invention provides a method comprising the steps of:
(S1) measuring the level of any one or more miRNAs selected from the group consisting of hsa-miR-11401, has-miR-12136, hsa-miR-145-3p, hsa-miR-2115-3p, hsa-miR-23a-3p, hsa-miR-27a-3p, hsa-miR-28-3p, hsa-miR-331-3p, hsa-miR-4435, hsa-miR-4507, hsa-miR-4685-3p, hsa-miR-501-3p, hsa-miR-618, and hsa-miR-7109-3p in a biological sample isolated from a subject;
(S2) comparing the level of the miRNA with the level of the miRNA in a biological sample isolated from a normal control group;
(S3) diagnosing a subject as having PD, when among the miRNAs in a biological sample isolated from the subject:
(a) the level of one or more miRNAs selected from the group consisting of hsa-miR-11401, hsa-miR-28-3p, hsa-miR-331-3p, hsa-miR-4435, hsa-miR-4685-3p, and hsa-miR-501-3p is more than 2 fold change increase compared to the level of the miRNAs in a biological sample isolated from a normal control group, and the AUC value is ≥0.5, the p-value is <0.5, the Confidence Limits value falls between 0.001 and 165; or
(b) the level of one or more miRNAs selected from the group consisting of hsa-miR-145-3p, hsa-miR-23a-3p, hsa-miR-27a-3p, and hsa-miR-618 is more than 2 fold change decreased compared to the level of the miRNAs in a biological sample isolated from a normal control group, and the AUC value is >0.5, the p-value is <0.5, the Confidence Limits value falls between 0.001 and 165; and
(S4) treating the PD with chemotherapy, surgery, or biological therapy when PD is diagnosed in step (S3), but not limited thereto.

### Advantageous Effects

According to a blood biomarker for diagnosis, prognosis prediction, or treatment of PD, a method for diagnosing PD, or predicting a prognosis using the same, and a method for screening substances for treating PD, it was confirmed that the expression levels of 14 types of miRNA were significantly increased or decreased in the blood of PD patients. In that the 14 types of miRNA reflect the correlation between genes and environmental factors identified in individuals with PD, the 14 types of miRNA are expected to be used as a diagnostic and prognostic biomarker as well as a new therapeutic target related to the pathological mechanism of PD.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIGS. 1A and 1B show the study workflow and sampling structure for differentially expressed genes (DEGs) and miRNAs (DEmiRNAs), respectively;

FIGS. 2A and 2B are heat maps showing differentially expressed genes (DEGs) and miRNAs (DEmiRNAs), respectively, in blood;

FIGS. 3A to 3D show a top-10 list of gene ontology and Kyoto Encyclopedia of Genes and Genomes (KEGG) pathways as a result of functional annotation and enrichment analysis of DEGs,

(FIG. 3A shows GO; biological processes, FIG. 3B shows GO; molecular functions; FIG. 3C shows GO; cellular components, and FIG. 3D shows KEGG pathways.);

FIG. 4 shows the results of integrated miRNA-mRNA analysis and refers to miRNA in blood (Here, significant miRNAs were selected when the p-value was less than 0.05. The red line represents a significance threshold value.);

FIGS. 5A and 5B show the receiver operating characteristic (ROC) curves of 14 types of miRNA identified in blood; and

FIGS. 6A to 6F show the results of evaluating the correlations of DEGs in the blood of PD patients with clinical factors that are known to affect the prognosis of PD, including fasting plasma glucose, serum insulin, insulin resistance (homeostasis model assessment of insulin resistance (HOMA-IR) score), uric acid, dietary pattern (Korean version of the Mediterranean diet adherence screener (K-MEDAS) score), and physical activity (physical activity scale for the elderly (PASE) score).

FIGS. 7A and 7B show the interaction network between significant miRNA-mRNA pairs in blood and FIG. 7C shows the DEmiRNAs interacting with the top 30 hub genes in blood.

### DETAILED DESCRIPTION OF EXEMPLARY EMBODIMENTS

The present invention provides a method, including the steps of:
(S1) measuring the level of any one or more miRNAs selected from the group consisting of hsa-miR-11401, has-miR-12136, hsa-miR-145-3p, hsa-miR-2115-3p, hsa-miR-23a-3p, hsa-miR-27a-3p, hsa-miR-28-3p, hsa-miR-331-3p, hsa-miR-4435, hsa-miR-4507, hsa-miR-4685-3p, hsa-miR-501-3p, hsa-miR-618, and hsa-miR-7109-3p in a biological sample isolated from a subject;
(S2) comparing the level of the miRNA with the level of the miRNA in a biological sample isolated from a normal control group.
(S3) diagnosing a subject as having Parkinson's disease (PD), when among the miRNAs in a biological sample isolated from the subject:
(a) the level of one or more miRNAs selected from the group consisting of hsa-miR-11401, hsa-miR-28-3p, hsa-miR-331-3p, hsa-miR-4435, hsa-miR-4507, hsa-miR-4685-3p, hsa-miR-501-3p, and hsa-miR-7109-3p is increased compared to the level of the miRNAs in a biological sample isolated from a normal control group; or
(b) the level of one or more miRNAs selected from the group consisting of hsa-miR-12136, hsa-miR-145-3p, hsa-miR-2115-3p, hsa-miR-23a-3p, hsa-miR-27a-3p, and hsa-miR-618 is decreased compared to the level of the miRNAs in a biological sample isolated from a normal control group; and
(S4) treating the PD with chemotherapy, surgery, or biological therapy when PD is diagnosed in step (S3), but not limited thereto.

Additionally, the present invention provides a method, including the steps of:
(S1) measuring the level of any one or more miRNAs selected from the group consisting of hsa-miR-11401, has-miR-12136, hsa-miR-145-3p, hsa-miR-2115-3p, hsa-miR-23a-3p, hsa-miR-27a-3p, hsa-miR-28-3p, hsa-miR-331-3p, hsa-miR-4435, hsa-miR-4507, hsa-miR-4685-3p, hsa-miR-501-3p, hsa-miR-618, and hsa-miR-7109-3p in a biological sample isolated from a subject;
(S2) comparing the level of the miRNA with the level of the miRNA in a biological sample isolated from a normal control group;
(S3) diagnosing a subject as having PD, when among the miRNAs in a biological sample isolated from the subject:
(a) the level of one or more miRNAs selected from the group consisting of hsa-miR-11401, hsa-miR-28-3p, hsa-miR-331-3p, hsa-miR-4435, hsa-miR-4685-3p, and hsa-miR-501-3p is more than 2 fold change increase compared to the level of the miRNAs in a biological sample isolated from a normal control group, and the AUC value is ≥0.5, the p-value is <0.5, the Confidence Limits value falls between 0.001 and 165; or
(b) the level of one or more miRNAs selected from the group consisting of hsa-miR-145-3p, hsa-miR-23a-3p, hsa-miR-27a-3p, and hsa-miR-618 is more than 2 fold change decreased compared to the level of the miRNAs in a biological sample isolated from a normal control group, and the AUC value is >0.5, the p-value is <0.5, the Confidence Limits value falls between 0.001 and 165; and
(S4) treating the PD with chemotherapy, surgery, or biological therapy when PD is diagnosed in step (S3), but not limited thereto.

In step (S4), the treatment of PD includes chemotherapy, surgery, or biological therapy selected from the group consisting of an anti-parkinsonian agent, therapeutic exercise, Deep Brain Stimulation (DBS), or the like, but is not limited to.

In the step of (S3) diagnosing the subject as having Parkinson's disease, the subject may be diagnosed as having Parkinson's disease if the |case/control fold change| is ≥2, preferably if the |case/control fold change| is >2.5, and most preferably if the |case/control fold change| is ≥3, but not limited thereto.

In the step of (S3) diagnosing a person as having Parkinson's disease, if the miRNA levels are altered, the subject can be diagnosed as having Parkinson's disease if the AUC value for determining the discriminatory power of the miRNA signature is ≥0.5, preferably ≥0.6 or ≥0.7, most preferably ≥0.8, but not limited thereto.

Further, in the above step of (S3) diagnosing a person as having Parkinson's disease, a subject may be diagnosed as having Parkinson's disease if the p-value is <0.5, preferably <0.4, more preferably <0.3, and most preferably <0.2, but not limited thereto.

Furthermore, during the above steps of (S3) diagnosing a subject as having Parkinson's disease, a person may be diagnosed as having Parkinson's disease if the Confidence Limits value falls between 0.001 and 165, but is not limited thereto.

The present invention provides a composition for diagnosing or predicting the prognosis of PD including, as an active ingredient, an agent for measuring the level of any one or more miRNAs selected from the group consisting of hsa-miR-11401, has-miR-12136, hsa-miR-145-3p, hsa-miR-2115-3p, hsa-miR-23a-3p, hsa-miR-27a-3p, hsa-miR-28-3p, hsa-miR-331-3p, hsa-miR-4435, hsa-miR-4507, hsa-miR-4685-3p, hsa-miR-501-3p, hsa-miR-618, and hsa-miR-7109-3p.

PD is the second most common neurodegenerative disease and primarily affects motor functions, including resting tremors, bradykinesia, rigididty, and loss of postural reflexes. The gold standard for diagnosing PD is confirming the loss of dopaminergic neurons and Lewy body pathology in the substantia nigra within the brain. However, the diagnosis of PD currently depends on clinical characteristics and neuroradiological features because available molecular biomarkers have not been identified.

The inventors of the present invention confirmed 14 types of miRNAs exhibiting a significant increase or decrease in expression levels in the blood of PD patients and normal controls. Furthermore, in that the 14 types of miRNAs of the present invention reflect the characteristics of PD patients that are influenced by environmental factors, it was confirmed that they are biomarkers with better accuracy as they indicate not only the presence or absence of gene expression but also the unique characteristics of PD.

In particular, although it was confirmed that the 14 types of miRNAs identified in the present invention do not necessarily require a combination of the 14 types for diagnosing or predicting the prognosis of PD or for screening substances for treating PD, a specific combination of the miRNAs may be used and each miRNA has accuracy for diagnosing PD, and whether an individual has PD may be diagnosed after confirming the expression level of any one of the 14 types of miRNAs from the blood of an individual suspected of having PD.

Therefore, the present invention provides a marker composition for diagnosing or predicting the prognosis of PD including, as an active ingredient, any one or more miRNAs selected from the group consisting of hsa-miR-11401, has-miR-12136, hsa-miR-145-3p, hsa-miR-2115-3p, hsa-miR-23a-3p, hsa-miR-27a-3p, hsa-miR-28-3p, hsa-miR-331-3p, hsa-miR-4435, hsa-miR-4507, hsa-miR-4685-3p, hsa-miR-501-3p, hsa-miR-618, and hsa-miR-7109-3p.

In particular, in that the level of SNCA, which is currently generally used for diagnosing or predicting the prognosis of PD, is not different in the blood of PD patients and normal controls, and thus cannot be distinguished, whereas the 14 types of miRNAs of the present invention exhibit an increase or decrease that is significantly different from the control group, and so the miRNAs may be used as a significant indicator to solve the difficulties involved in existing technologies for diagnosing or predicting the prognosis of PD patients.

In one embodiment of the present invention, the composition may be for diagnosing or predicting the prognosis of PD in a group in which the presence or absence of PD cannot be determined by a-synuclein, and furthermore, it may be for screening substances for prevention or treatment of this group, but is not limited thereto.

In one embodiment of the present invention, the level of miRNA may be measured from any one selected from the group consisting of blood, serum, whole blood, and plasma, but is not limited thereto.

In the present specification, “miR” or “miRNA” refer to a small non-coding RNA consisting of 18 to 25 nucleotides (nt), and it refers to a non-coding RNA consisting of 21 to 23 nt which binds to the 3′-untranslated region (UTR) of a target gene to enhance degradation thereof or inhibit translation thereof, thereby regulating gene expression. MiRNAs are processed from introns, exons, or intergenic regions. In general, miRNA is transcribed into a 70 to 80 nt long precursor with a hairpin structure called pre-miRNA, and then cleaved by dicer, an RNAse III enzyme, to be produced in a mature form. MiRNAs form a ribonucleoprotein complex, called miRNP, which cleaves a target gene or inhibits translation through complementary binding to a target site. More than 30% of human miRNAs are present in a cluster, which is transcribed into single precursors, and then the final mature miRNAs are formed through a cleavage process. An miRNA has a structure such as a hair pin, and the structure is cut and divided. An miRNA originating from the left arm of the hairpin is denoted with a 5p suffix, and an miRNA originating from the right arm is denoted with a 3p suffix.

In the present invention, “an agent for measuring the level of an miRNA” may be a primer, probe, oligonucleotide, antibody or antigen-binding fragment thereof, ligand, aptamer, peptide, receptor, agonist or antagonist that specifically binds to an mRNA, or a combination thereof, but is not limited thereto, and it may refer to any agent commonly used in the art.

The purpose of the present invention is to analyze the diagnosis, prognosis prediction, and screening of PD through a significant increase or decrease of 14 types of miRNA, and the feature of the present invention is the type of miRNA subject to analysis and the pattern of increase or decrease depending on the type, not the type of agent for measuring the level of the above-described miRNA.

According to one embodiment of the present invention, 14 types of miRNAs were identified from blood isolated from PD patients: eight types of miRNAs among the 14 types of miRNAs were significantly increased (Nos. 1, 7 to 12, and 15 in Table 4 of the present invention); and six types of miRNA were significantly decreased (Nos. 2 to 6 and 13 in Table 4 of the present invention).

Therefore, the present invention provides a method for providing information for diagnosing or predicting the prognosis of PD, including:
(S1) measuring the level of any one or more miRNAs selected from the group consisting of hsa-miR-11401, has-miR-12136, hsa-miR-145-3p, hsa-miR-2115-3p, hsa-miR-23a-3p, hsa-miR-27a-3p, hsa-miR-28-3p, hsa-miR-331-3p, hsa-miR-4435, hsa-miR-4507, hsa-miR-4685-3p, hsa-miR-501-3p, hsa-miR-618, and hsa-miR-7109-3p in a biological sample isolated from a subject; and
(S2) comparing the level of the miRNA with the level of the miRNA in a biological sample isolated from a normal control group.

In one embodiment of the present invention, the method for providing information may further include (S3) diagnosing a subject as having PD, when among the miRNAs in a biological sample isolated from the subject,
(a) the level of one or more miRNAs selected from the group consisting of hsa-miR-11401, hsa-miR-28-3p, hsa-miR-331-3p, hsa-miR-4435, hsa-miR-4507, hsa-miR-4685-3p, hsa-miR-501-3p, and hsa-miR-7109-3p is increased compared to the level of the miRNAs in a biological sample isolated from a normal control group; or
(b) the level of one or more miRNAs selected from the group consisting of hsa-miR-12136, hsa-miR-145-3p, hsa-miR-2115-3p, hsa-miR-23a-3p, hsa-miR-27a-3p, and hsa-miR-618 is decreased compared to the level of the miRNAs in a biological sample isolated from a normal control group, but is not limited thereto.

In the present invention, a “biological sample” may include anything without limitation as long as it is taken from a subject for whom PD is to be diagnosed or the prognosis thereof is to be predicted.

In one embodiment of the present invention, the biological sample may be any one selected from the group consisting of blood, serum, whole blood, and plasma, but is not limited thereto.

The biological sample may be pretreated before it is to be used for detection or diagnosis. For example, the pretreatment may include homogenization, filtration, distillation, extraction, concentration, inactivation of interfering ingredients, addition of reagents, etc. The sample may be prepared to increase the detection sensitivity of a protein marker, for example, a sample obtained from a subject may be pretreated using methods such as anion exchange chromatography, affinity chromatography, size exclusion chromatography, liquid chromatography, sequential extraction or gel electrophoresis.

The term “method for providing information” used in the present invention refers to a method of providing information regarding the diagnosis of a disease, and it refers to a method of acquiring information about the onset or possibility (risk) of developing a disease by analyzing a biological sample of an individual or confirming an increase or decrease in the level of a biomarker according to the present invention. For example, as the method for providing information is a method of measuring the level of a biomarker according to the present invention and comparing it with a control group to provide information on whether to diagnose an individual as having PD or whether the prognosis prediction is good or bad, through the method, it is possible to diagnose PD, prescribe the necessary treatment method, and predict the therapeutic effect of the treatment, through appropriate treatment for PD.

In the present specification, the expression “the level is increased” means that something that was not detected has been detected, or that the amount of detection is relatively higher than the normal level. For example, an “increased” level means that the level in the experimental group is at least 1%, 2%, 3%, 4%, 5%, 10% or more, for example, 5%, 10%, 20%, 30%, 40%, or 50%, 60%, 70%, 80%, 90% or higher than that of the control group, and/or 0.5 times, 1.1 times, 1.2 times, 1.4 times, 1.6 times, 1.8 times or higher that of the control group. Specifically, it may mean that the level is 1 to 1.5 times, 1.5 to 2 times, 2 to 2.5 times, 2.5 to 3 times, 3 to 3.5 times, 3.5 to 4 times, 4 to 4.5 times, 4.5 to 5 times, 5 to 5.5 times, 5.5 to 6 times, 6 to 6.5 times, 6.5 to 7 times, 7 to 7.5 times, 7.5 to 8 times, 8 to 8.5 times, 8.5 to 9 times, 9 to 9.5 times, 9.5 to 10 times, or 10 times or more that of the control group, but is not limited thereto. In addition, in the present invention, the expression that “the expression level of miRNA is decreased” may have a broad meaning including a statistically significant level, but is not limited to thereto. The meaning of the opposite term may be understood by those skilled in the art as having the opposite meaning according to the above definition.

In the present invention, when the term “including” is used, unless otherwise specified, it means that other components may be further included rather than excluding other components. The term “step of . . . ” used throughout the present invention does not mean “step for.”

The present invention provides a method for screening substances for preventing or treating PD, including a step of measuring the level of any one or more miRNAs selected from the group consisting of hsa-miR-11401, has-miR-12136, hsa-miR-145-3p, hsa-miR-2115-3p, hsa-miR-23a-3p, hsa-miR-27a-3p, hsa-miR-28-3p, hsa-miR-331-3p, hsa-miR-4435, hsa-miR-4507, hsa-miR-4685-3p, hsa-miR-501-3p, hsa-miR-618, and hsa-miR-7109-3p in a biological sample isolated from a PD animal model to which a candidate substance was administered.

In one embodiment of the present invention, the method for screening may further include a step of selecting the candidate substance as a substance for PD prevention or treatment, when among the miRNAs in a biological sample isolated from the subject,
(a) the level of one or more miRNAs selected from the group consisting of hsa-miR-11401, hsa-miR-28-3p, hsa-miR-331-3p, hsa-miR-4435, hsa-miR-4507, hsa-miR-4685-3p, hsa-miR-501-3p, and hsa-miR-7109-3p is increased compared to the level of the miRNAs in a biological sample isolated from a normal control group; or
(b) the level of one or more miRNAs selected from the group consisting of hsa-miR-12136, hsa-miR-145-3p, hsa-miR-2115-3p, hsa-miR-23a-3p, hsa-miR-27a-3p, and hsa-miR-618 is decreased compared to the level of the miRNAs in a biological sample isolated from a normal control group, but is not limited thereto.

In the present invention, in that 14 types of miRNAs exhibiting a significant difference in an increase or decrease in the blood between the control group and the PD group were confirmed, it was confirmed the 14 types of miRNAs are not only biomarkers for diagnosing or predicting the prognosis of PD but also targets for treatment, that is, therapeutic biomarkers of PD. Therefore, when developing a therapeutic agent for PD, an agent capable of regulating (increasing or decreasing) the level of the 14 types of miRNAs of the present invention may be confirmed to be a therapeutic substance for PD through an in vivo or in vitro experiment, and so it may be used for screening purposes.

In the present invention, “screening” may refer to selecting a substance with specific properties from a candidate group consisting of various substances using a specific manipulation or evaluation method.

In the present invention, the term “treatment” refers to all actions that improve or beneficially change a target disease and metabolic abnormalities thereof, and methods such as chemotherapy, surgery, or biological therapy may be used. The term may be used interchangeably with “improvement,” but is not limited thereto.

According to the purpose of the present invention, the screening method of the present invention may refer to a series of processes including the step of determining the effectiveness of a drug candidate substance by the above method to identify a therapeutic agent that produces the best therapeutic effect for patients with PD, but is not limited thereto.

The step of confirming the treatment response and effect may be repeated several times depending on the therapeutic candidate, and additional steps used in the art as general screening methods such as adding additional substances or steps to confirm the treatment response and effect, may be additionally included, but are not limited thereto.

In the present invention, “candidate substance” refers to an unknown substance used in screening to prevent or treat PD by increasing or decreasing the expression of the 14 types of miRNAs of the present invention by administering it to a PD animal model, includes all formulations and methods that can be used to treat various types of PD, and it may be one or more selected from the group consisting nucleotides, DNA, RNA, amino acids, aptamers, proteins, stem cells, stem cell culture solutions, compounds, microbial cultures or extracts, natural products, and natural extracts, but is not limited thereto.

At this time, when the substance increases or decreases the level of the miRNA depending on the type compared to a comparative group, for example, a positive control group, a negative control group (normal control in the present invention), etc., it may be determined that PD is treated (or improved). In the present specification, the meaning of the expression “the level is increased” is the same as described above.

In the present invention, although substances or methods that are commonly used in the art to treat PD are applied, additionally, commonly used treatment methods may be used, or drugs that are commonly used to assist in the treatment of PD may be further administered, but are not limited thereto.

The present invention provides a kit for diagnosing or predicting the prognosis of PD, including an agent for measuring the level of any one or more miRNAs selected from the group consisting of hsa-miR-11401, has-miR-12136, hsa-miR-145-3p, hsa-miR-2115-3p, hsa-miR-23a-3p, hsa-miR-27a-3p, hsa-miR-28-3p, hsa-miR-331-3p, hsa-miR-4435, hsa-miR-4507, hsa-miR-4685-3p, hsa-miR-501-3p, hsa-miR-618, and hsa-miR-7109-3p; and instructions.

In one embodiment of the present invention, the instructions may teach the method for providing information, but are not limited thereto.

In the present invention, “kit” refers to a tool further including substances for preparation or function, storage, or the like that enable the purpose of kit claimed in the present invention to be performed. In addition to the above substances, the kit of the present invention may include other components, compositions, solutions, devices, and the like commonly required for storage and processing methods. As a specific example, at this time, each component may be applied one or more times without limit, there is no limit in the sequence of application of each material, and the application of each material may be carried out simultaneously or at different times.

In the present invention, the kit may include a container; instructions; or the like. The container may serve to package the substances, and may also serve to store and secure them. The material of the container may be in the form of, for example, a bottle, a tub, a sachet, an envelope, a tube, an ampoule, or the like, which may be partially or entirely made of plastic, glass, paper, foil, wax, or the like. The container may be equipped with a completely or partially removable cover that may initially be part of the container or may be attached to the container by mechanical, adhesive, or other means, or a stopper that may provide access to the contents by a needle may be mounted. The kit may include an external package, and the external package may include instructions for use of the components.

In addition, the present invention may provide a diagnostic device for diagnosing, predicting a prognosis, and even treating PD in an individual. A measurement unit of the diagnostic device of the present invention may use an agent for measuring the level of the 14 types of miRNAs, which are biomarkers according to the present invention, to measure the expression level of the miRNAs in a biological sample (e.g., blood, etc.) obtained from a subject. By measuring the expression level of the protein or gene using the agent in the measurement unit, PD may be diagnosed, and the treatment may be diagnosed as having a high treatment effect.

The diagnostic device of the present invention may further include a detection unit that predicts and outputs the presence, disease stage, or type of PD in a subject based on the expression level of the protein or gene obtained in the measurement unit.

In the present invention, the detection unit may determine the implementation of the treatment by generating and classifying information on the diagnosis or prognosis prediction of PD according to the category of the expression level of the protein or gene obtained in the measurement unit.

In the present invention, since “detection” may include quantifying the concentration of a target and include a qualitative meaning (qualitative analysis) of confirming the presence or absence of a specific substance, it has a meaning including both measuring and confirming the presence (expression) of a target substance or measuring and confirming changes in the level of presence (expression level) of the target substance.

In the present invention, “diagnosis” refers to confirming the presence or characteristics of a pathological condition. For the purpose of the present invention, diagnosis is not limited to confirming the diagnosis of PD, but it also includes predicting the prognosis of PD. More illustratively, the term used in the present invention, “diagnosis,” refers to determining the susceptibility of a subject to a specific disease or disorder, determining whether a subject currently has a specific disease or disorder, determining the prognosis (e.g., identifying a tumor state, determining the stage of a tumor, or determining the responsiveness of cancer to treatment, in the present invention, diagnosis of PD or progression of the disease) of a subject with a specific disease or disorder, or therametrics (e.g., monitoring the conditions of a subject to provide information about treatment efficacy).

In the present invention, “prognosis prediction” may refer to predicting the degree of disease progression when an individual diagnosed as having a disease is treated with a specific therapeutic substance. In other words, in the present invention, it may mean predicting the probability of progression, worsening, recurrence, maintenance, etc. of PD through the increase or decrease in the level of a biomarker of the present invention.

In the present invention, “measurement” has a meaning including detecting and confirming the presence (expression) of a target substance, or detecting and confirming a change in the level of presence (expression level) of a target substance. The measurement may be performed without limitation and includes both qualitative methods (analysis) and quantitative methods. The types of qualitative methods and quantitative methods for measuring the presence/level of a biomarker of the present invention are well known in the art and include the experimental methods described in the present specification.

The term “confirmation” used in the present specification may correspond to the meaning of “analysis,” but is not limited thereto. “Analysis” may preferably refer to “measurement,” and the qualitative analysis may refer to measuring and confirming the presence of a target substance, and the quantitative analysis may refer to measuring and confirming a change in the level of presence (expression level) or amount of a target substance.

In the present invention, analysis or measurement may be performed without limitation and includes both qualitative methods and quantitative methods, and quantitative measurement may be preferably performed.

In the present invention, “determination” may be used interchangeably with “discrimination” and refer to distinguishing a subject according to a specific standard. In the present invention, it may be used to refer to predicting the prognosis of an individual diagnosed as having or likely to have PD, but is not limited thereto.

In the present invention, “subject” means a target that requires disease risk prediction, diagnosis, prognosis prediction, or treatment, and more specifically, it may refer to human or non-human primates, mammals such as mice, rats, dogs, cats, horses, and cows, but is not limited thereto.

In the present invention, “administration” refers to providing a predetermined composition of the present invention to an individual by any appropriate method.

The terms used in the present invention are selected as general terms that are currently widely used as much as possible while considering the functions in the present invention, but they may vary depending on the intention or precedent of those of ordinary skill in the art, the emergence of new technology, or the like. In addition, in certain cases, there are terms arbitrarily selected by the applicant, and in this case, the meaning will be described in the corresponding part of the detailed description of the invention. Therefore, the terms used in the present invention should be defined based on the meaning of the terms and the overall content of the present invention, rather than simply the names of the terms.

Hereinafter, preferred examples are presented to aid understanding of the present invention. However, the following examples are provided only to make the present invention easier to understand, and the content of the present invention is not limited by the following examples.

### EXAMPLES

### Participants

The study protocol was approved by the Institutional Review Board of the Catholic University of Korea Seoul St. Mary's Hospital (IRB approval number KC22TISI0415). All participants provided written consent prior to their participation.

This study included seven patients newly diagnosed with PD according to the UK Brain Bank criteria and a control group consisting of six subjects who underwent SMG resection for head and neck cancer at the Department of Neurology and the Department of Otolaryngology-Head and Neck Surgery at the Catholic University of Korea Seoul St. Mary's Hospital between October 2022 and April 2023 without age-related, gender-related, neurological or psychological disorders. The patient exclusion criteria were as follows: (1) young onset PD (age of onset of 40 years or less), (2) family history of PD, (3) normal dopamine transporter scan according to Movement Disorder Society's clinical diagnostic criteria for PD, (4) neurological or neuroradiological abnormalities associated with atypical PD or secondary PD, (5) abnormalities of the basal ganglia or cerebellum found in structural magnetic resonance imaging, (6) use of an anticoagulant, (7) known SMG disease, (8) elderly people (people over 75 years of age who may be vulnerable to invasive procedures), and (9) severe dementia. Medical record review and neurological examination were performed on the control subjects to ensure that each individual was free from neurological or psychological disorders, including idiopathic rapid eye movement (REM) sleep behavior disorder, essential tremor, PD, dementia, and depression.

Clinical information on age, gender, body mass index (BMI), hypertension, diabetes, smoking habits, and dietary habits was collected using the K-MEDAS questionnaire, and information on physical activity habits was collected using the PASE questionnaire for the PD patients and controls. In addition, disease duration, a Unified Parkinson's disease Rating Scale (UParkinsonRS) score, and a modified Hoehn and Yahr (H&Y) staging score of the PD patients were determined.

### Sample Collection Procedure

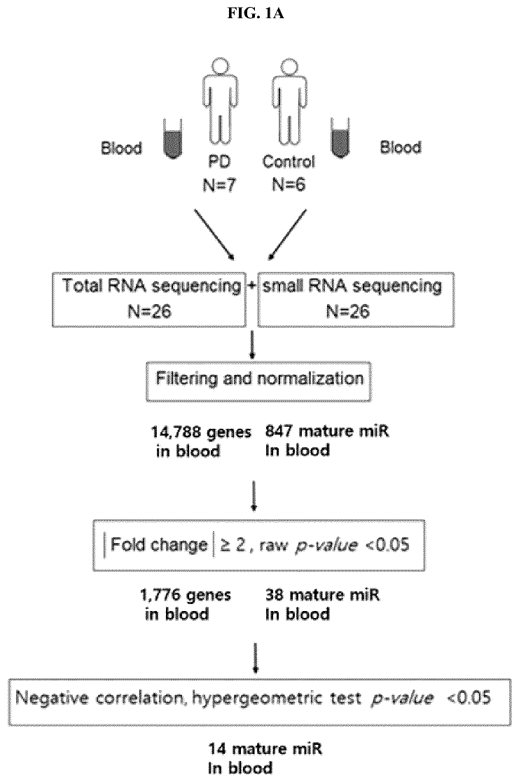

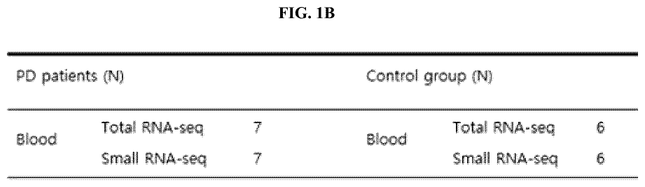

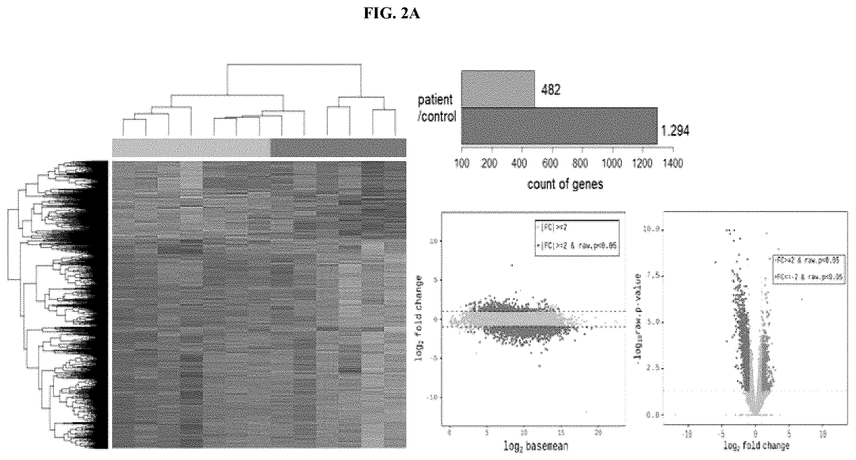

The study workflow is shown in FIG. 1A. Approximately 3 mL of whole blood was obtained from each PAXgene Blood RNA Tube, and a total of 2 PAXgene Blood RNA Tubes were collected from each participant. One tube was for transcriptome sequencing, and the other tube was for smRNA-seq. Clinical blood samples were collected simultaneously to measure fasting plasma glucose, serum insulin, hemoglobin A1c (HbA1c), C-peptide, and lipid profiles for the PD patients and controls. Fasting was defined as abstinence from caloric intake for at least eight hours.

### Sample Preparation and RNA-Seq

RNA-seq was performed on the blood samples (n=52; 26 total RNA, 26 smRNA) from all the study participants (FIGS. 1A and 1B). RNA was extracted from the blood samples according to the PAXgene Blood RNA kit instructions. All samples had an RNA integrity number (RIN)≥6 except for one blood sample in which RIN=4.9 (mean RIN=7.88, range of 4.9 to 8.8). All samples were included for cDNA library construction.

The libraries were prepared using the Illumina TruSeq Standard Total RNA Library Prep Globin Kit (Illumina, Inc.) for the blood and the Illumina TruSeq Small RNA Library Prep Kit (Illumina, Inc.) for the blood samples. The total RNA library was validated using TapeStation D1000 ScreenTape (Agilent Technologies, Santa Clara, USA), and the smRNA library was assessed for size, purity, and concentration using an Agilent Bioanalyzer (Agilent Technologies). A validated index library was submitted for sequencing on the Illumina NovaSeq™ 6000 (Illumina, Inc.). Paired-end (2×100 bp) sequencing was performed on the total RNA-seq library for transcriptome analysis, and single-end (51 bp) sequencing was performed on the smRNA-seq library for miRNA analysis.

### RNA-Seq Data Analysis

As a computational analysis method, raw read sequencing data (FASTQ) was applied for quality assessment of reads, trimming, alignment, normalization, expression, and functional annotation. For total RNA-seq, 3′ adapter sequences and low-quality reads were removed using Trimomatic 0.38 (http://www.usadellab.org/cms/?page=trimmomatic). Trimmed reads were mapped to the human GRCh38 (hg38) reference genome (annotation NCBI_109.20200522) using HISAT2 (https://ccb.jhu.edu/software/hisat2/index.shtml). Expression profiling was performed with the mapped reads using StringTie (https://ccb.jhu.edu/software/stringtie/). For each gene, fragments per kilobase of transcripts per million (FPKM) and transcripts per million (TPM) were obtained. After filtering out underexpressed genes using one or more zero counts, the remaining reads were normalized using the Relative Log Expression (REL) method implemented in the DEseq2 package in the R library (http://www.bioconductor.org/). Differentially expressed genes between the PD patients and controls were estimated by the negative binomial Wald test using DEseq2. Criteria for significant results were |case/control fold change|≥2 and raw p-value<0.05.

Gene ontology (GO) enrichment analysis and functional annotation were performed on differentially expressed genes (DEGs) using the g: Profiler tool (https://biit.cs.ut.ee/gprofiler/). The relevant biological pathways associated with the DEGs were analyzed using the Kyoto Encyclopedia of Genes and Genomes (KEGG) pathway database (http://www.kegg.jp/kegg/pathway.html). For smRNA-seq, Cutadapt 2.8 (https://cutadapt.readthedocs.org/en/stable/) was used for trimming. The finally processed reads sequentially aligned to the reference genome, miRBase v22.1 (http://www.mirbase.org/). Genome mapping was processed with Bowtie (http://bowtie-bio.sourceforge.net/index.shtml) and Spliced Transcripts Alignment to a Reference (STAR) using RNA-Seq by Expectation-Maximization (RSEM) (http://deweylab.github.io/RSEM/). Known/novel miRNAs were predicted using miRDEEP2 (https://www.mdc-berlin.de/content/mirdeep2-documentation). After excluding mature miRNAs with zero counts across more than 51% of all samples, read count data was normalized using a trimmed average M-value (TMM) method in the edgeR library (https://bioconductor.org/packages/3.17/bioc/). Differentially expressed miRNAs were analyzed by an exact test using edgeR, and significance was determined based on |case/control fold change|≥2 and raw p-value<0.05.

Hierarchical clustering through Euclidean distance and complete linkage grouped the similarities of the genes and the mature miRNAs based on the normalized expression levels of the significant list. miRNA-mRNA integration analysis extracted the differentially expressed mRNAs and miRNAs between the compared samples, and a negative correlation of the target pairs between mRNAs and miRNAs was confirmed based on miRDB v6.0 (http://mirdb.org/miRDB/). The p-value was derived from a hypergeometric test for the mRNA targeted by the miRNA. The sample preparation, next-generation sequencing (NGS), and computational sequencing analysis were performed at Macrogen Inc. (Seoul, Korea).

### Statistical Analysis

Descriptive statistics were presented as mean±standard deviation (SD) or frequency for each clinical characteristic. Analysis was performed using the independent t-test for continuous variables and the Fisher's exact test for categorical variables. Pearson's correlation coefficient was used to determine the correlation between DEGs and clinical factors, including fasting plasma glucose, serum insulin, HOMA-IR score, serum uric acid, K-MEDAS score, and PASE score. As the normalized expression values of DEGs, the TPM value for each gene was used. For the PD patients, the covariates of age, gender, BMI, and disease duration were controlled, and for the control group, age, gender, and BMI were controlled.

A binomial logistic regression model was fit to investigate the diagnostic ability of the identified miRNAs based on an integrated analysis of miRNAs, excluding has-miR-12136 in the blood due to complete separation. The predicted probability was calculated based on receiver-operating characteristic (ROC) curve and area under the curve (AUC) analysis, excluding has-miR-12136 in the blood. The normalized expression values for each miRNA were calculated using TMM. The difference between multiple ROC curves and AUC values of miRNAs was compared using Wilcoxon-Mann-Whitney U statistics.

All clinical statistical analyses were performed using SAS (version 9.4; SAS Institute Inc., Cary, NC, USA) statistical software. A p-value<0.05 was considered statistically significant.

Construction of the protein-protein interaction (PPI) network analysis was performed using the Search Tool for the Retrieval of Interaction Genes (STRING) database (version 12.0) (http://www.string-db.org/) with confidence threshold of 0.2. Subsequently, the hub genes in the interaction network were identified by analyzing the degree of connectivity using Cytoscape (version 3.10.2) and the cytoHubba plugin (version 0.1). To visualize the interaction network of target genes with differentially expressed miRNAs (DEmiRNAs), Cytoscape (version 3.10.2) was also employed.

### Example 1. Clinical Characteristics

A total of 52 samples were collected from seven PD patients and six controls. Of these samples, 28 were from the PD patients and 24 were from the controls (FIGS. 1A and 1B). The clinical characteristics of the PD patients and controls are summarized in Table 1. The patients and controls recruited for this study were matched for age (PD patients vs. controls; 64.1±6.1 vs. 61.7±6.1, p=0.4792) and gender (p=1). There was no significant difference between the PD patients and controls in the baseline characteristics of BMI, smoking history, hypertension, diabetes, fasting plasma glucose, glycated hemoglobin (HbA1c), serum insulin, C-peptide, HOMA-IR score, uric acid, and K-MEDAS score. The mean disease duration of the PD patients was 17±14.0 months, and a significant difference was observed in the physical activity score between the PD patients and controls (PASE score, PD patients vs. controls, 204.2±60.8 vs. 95.9±49.6; p=0.0046).

TABLE 1

| Item | Patients | | Controls | | p-value |
|---|---|---|---|---|---|
| N | 7 | | 6 | | |
| Age, year | 64.1 ± 6.1 | | 61.7 ± 6.1 | | 0.4792 |
| Sex, n (%) | | | | | 1.0000 |
| Men | 4 | (57.1%) | 3 | (50%) | |
| Women | 3 | (42.9%) | 3 | (50%) | |
| BMI, kg/m² | 24.9 ± 3.8 | | 24.7 ± 6.5 | | 0.9475 |
| Smoker, n (%) | | | | | 0.5594 |
| Non-smoker | 4 | (57.1%) | 5 | (83.3%) | |
| Ex-smoker | 3 | (42.9%) | 1 | (16.7%) | |
| Hypertension, n (%) | 2 | (28.6%) | 4 | (66.7%) | 0.2861 |
| Diabetes mellitus, n (%) | 0 | (0%) | 2 | (33.3%) | 0.1923 |
| Fasting plasma glucose | 106 ± 15.9 | | 135.8 ± 34.7 | | 0.0649 |
| (mg/dL) | | | | | |

| | | | |
|---|---|---|---|
| HbA1c(%) | 5.7 ± 0.2 | 5.9 ± 0.5 | 0.3341 |
| Insulin (uIU/mL) | 10.1 ± 7.6 | 7.42 ± 4.1 | 0.4604 |
| C-peptide(ng/mL) | 2.1 ± 0.96 | 2.3 ± 0.68 | 0.5999 |
| HOMA-IR score | 2.6 ± 1.9 | 2.4 ± 1.3 | 0.8042 |
| Uric acid (mg/dL) | 4.6 ± 1.3 | 4.7 ± 1.4 | 0.8501 |
| Disease Duration, month | 17 ± 14.0 | N/A | |
| UPDRS part III | 30.4 ± 18.6 | N/A | |
| UPDRS total | 42.1 ± 20.4 | N/A | |
| H & Y stage | 1.9 ± 1.1 | N/A | |
| K-MEDAS score | 7.6 ± 2.1 | 6.8 ± 3.4 | 0.6416 |
| PASE score | 95.9 ± 49.6 | 204.2 ± 60.8 | 0.0046* |

### Example 2. Differential Expression of mRNA and miRNA in PD Patients

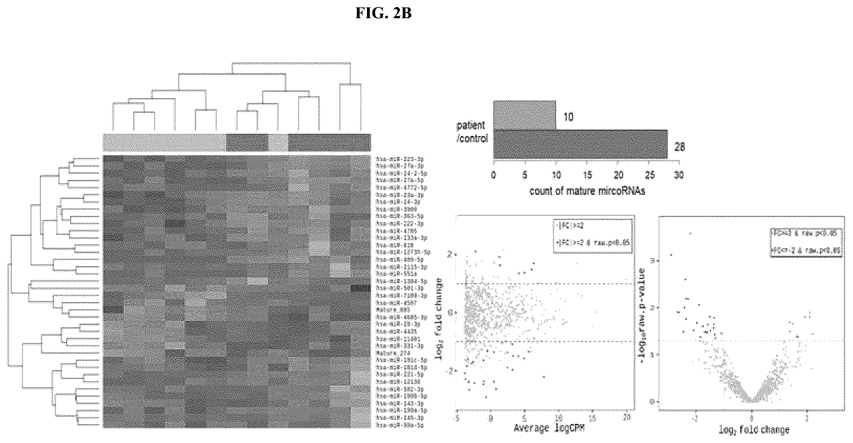

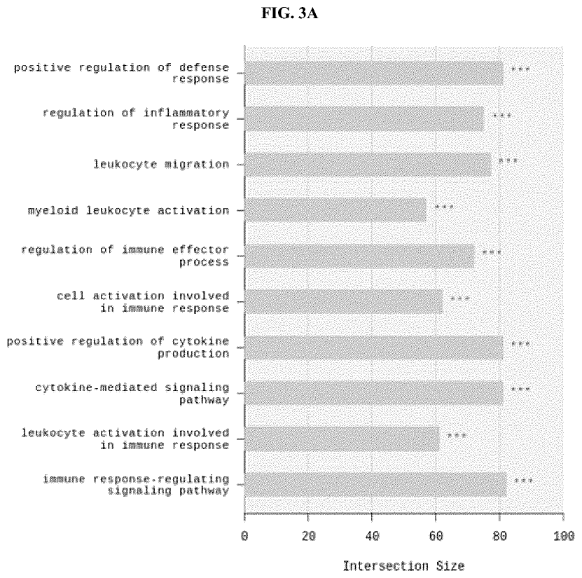

The whole transcriptome sequencing data and smRNA-seq data were deposited in the National Center for Biotechnology Information (NCBI) (Sequence Read Archive SRA) database (BioProject accession number: PRJNA1027508). After filtering out underexpressed genes and miRNAs, 14,788 genes in the blood and 847 mature miRNAs in the blood were processed for differential expression analysis. A total of 1,776 genes and 38 miRNAs in the blood showed significant differential expression in the PD patients (FIG. 1A). Heatmap visualization shows the up-regulated and down-regulated differentially expressed genes (DEGs) (FIG. 2A) and the differentially expressed miRNAs (FIG. 2B), and the dendrogram shows the hierarchical clusters.

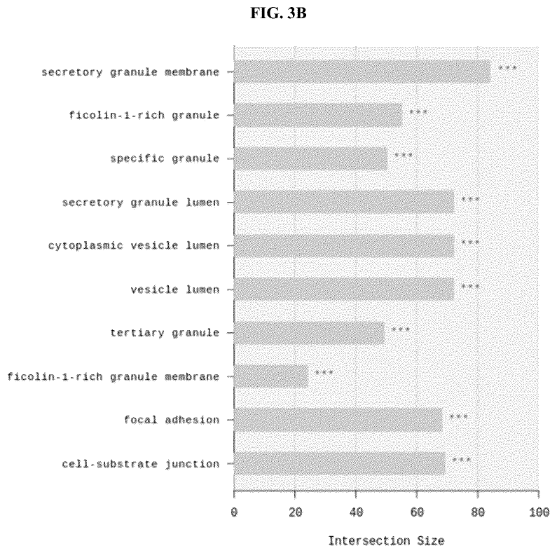

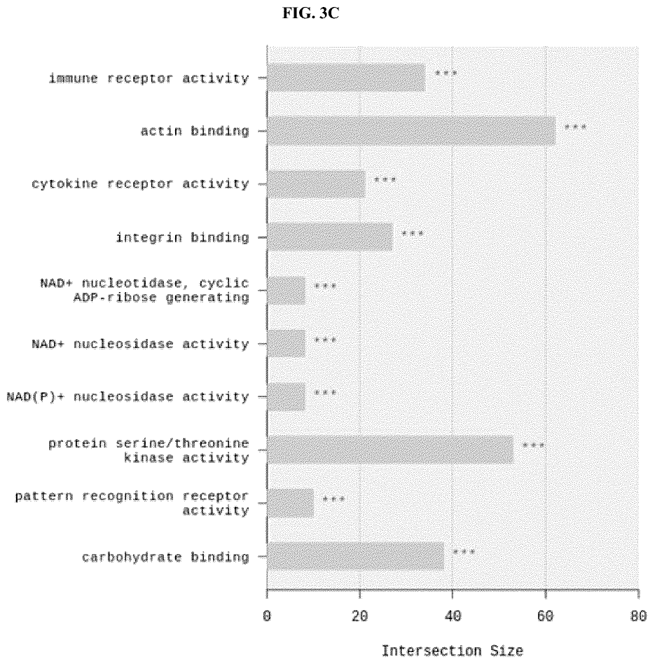

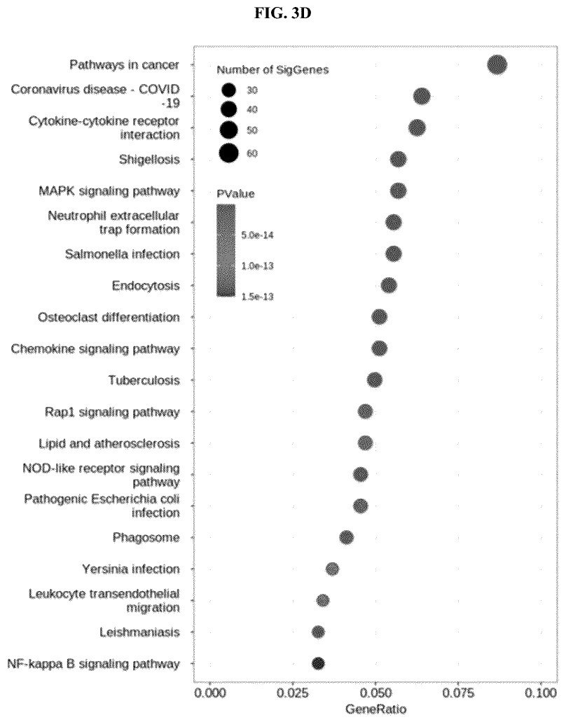

The functional annotation and enrichment analysis results of the DEGs are shown in FIGS. 3A to 3D. Most of the highest terms in the gene ontology (GO) analysis of the blood samples were cytokine-related inflammation, immune responses, cell membrane, or secretory granule-related functions (FIGS. 3A to 3C). Viral or other infection-related pathways were the highest interacting pathways in the KEGG analysis (FIG. 3D).

Since the filtering criterion was |case/control fold change|≥2, the expression of SNCA-related genes, which are genes encoding the α-synuclein protein and which were filtered out due to differential expression below the threshold, were carefully examined.

No significant difference was found in the expression associated with the SNCA gene between the PD patients and controls in the blood samples.

### Example 3. Prediction of Functional Interaction Between miRNA and Target Gene

To analyze the functional roles of the differentially expressed miRNAs in the blood samples from the PD patients, miRbase version 22, which is an miRNA database for miRNA gene annotation, was utilized. Because miRNA induces the degradation or destabilization of mRNA and changes its expression level, a negative correlation is predicted when miRNA-mRNA functionally interact. Predicted mRNA targets of the differentially expressed miRNAs were extracted from the database, and miRNAs exhibiting a negative correlation with actual gene expression in the samples were selected. Through this, the actual regulation of gene expression in the PD patients was confirmed rather than relying only on in silico predictions. The significance of miRNA-mRNA negative correlation pairs was calculated using a hypogeometric test. Significant miRNAs were selected when the p-value was <0.05.

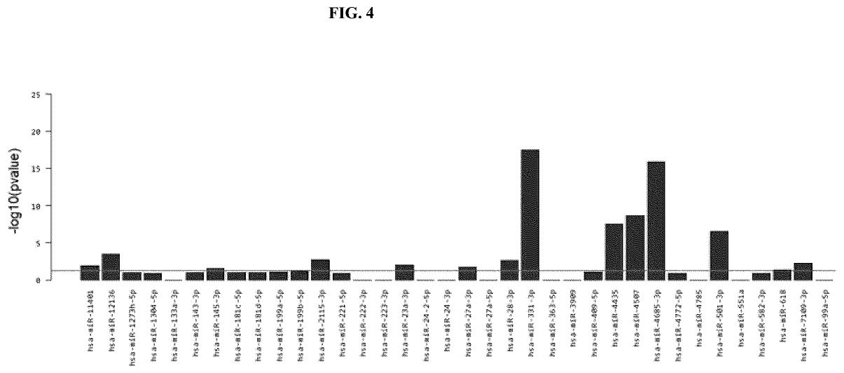

As a result, among the differentially expressed miRNAs in the blood (38 miRNAs), 14 significant mature miRNAs showed a negative correlation with the target genes expressed in each sample type (FIGS. 1A and 4).

### Example 4. Comparison of Tissue-Specific Transcriptional Regulation and Diagnostic Ability of miRNA Signatures for PD Between SMG and Blood

Among the DEGs and miRNAs in the blood of the PD patients, the number of miRNAs was quantified. Only one out of the 1,776 DEGs in the blood was down-regulated in the PD patients (Table 2). It was found that only two genes (THBS1 and NFAM1) were regulated by hsa-miR-331-3p in the blood (Table 3).

TABLE 2

| | Common gene | | |
|---|---|---|---|
| MicroRNA | symbol | P/C FC | raw p-value |
| | BCL2A1 | −4.037918 | 0.032645647 |
| | C4A | −2.695161 | 0.000710347 |
| | C4B | −2.814209 | 0.000528877 |
| | CEBPD | −3.827470 | 0.000109569 |
| | CCR1 | −2.987953 | 7.81833E−06 |
| | PLK3 | −2.403917 | 0.001557917 |
| hsa-miR-12136 | CR1 | −5.922396 | 9.90477E−08 |
| hsa-miR-27a-3p | CYP1B1 | −4.171870 | 3.83913E−05 |
| hsa-miR-12136 | DUSP1 | −3.079049 | 0.000712624 |
| hsa-miR-12136 | GPR183 | 2.288184 | 0.010415659 |
| hsa-miR-12136 | EMP1 | 3.803685 | 3.3972E−06 |
| | FCGR3B | −2.396745 | 0.004103045 |
| | FCN1 | −2.042030 | 1.27689E−05 |
| hsa-miR-501-3p | FOS | −2.994268 | 0.000771862 |
| | FPR1 | −3.202315 | 1.69422E−06 |
| | CXCL1 | −2.411925 | 0.000244231 |
| | CFH | 2.181260 | 0.020916315 |
| | ICAM1 | −2.276567 | 0.001349 |
| | IL1B | −3.357036 | 1.1889E−05 |
| | IL7R | 2.440283 | 1.18016E−05 |
| | ITGB3 | −2.613583 | 0.003007794 |
| hsa-miR-11401, | JUNB | −3.759492 | 4.13784E−06 |
| hsa-miR-12136 | | | |
| hsa-miR-4507 | GADD45B | −2.746131 | 4.26707E−05 |
| hsa-miR-27a-3p | NFIL3 | −3.113215 | 0.000385667 |
| | CFP | −2.239263 | 0.000141207 |
| | SRGN | −2.150202 | 0.0416045 |
| | S100A9 | −3.865474 | 0.00027415 |
| | SELL | −2.795192 | 0.000296918 |
| | SELP | −2.092733 | 0.001719843 |
| | SLA | −2.703023 | 0.000225285 |
| hsa-miR-4435, | SLC2A3 | −4.644082 | 1.85642E−07 |
| hsa-miR-4685-3p | | | |
| | SLC11A1 | −3.746816 | 2.15278E−05 |
| | THBD | −3.830043 | 0.000103441 |
| hsa-miR-331-3p, | THBS1 | −3.114932 | 1.09916E−07 |
| hsa-miR-4685-3p, | | | |
| hsa-miR-618 | | | |
| hsa-miR-27a-3p | ZFP36 | −3.072648 | 7.32945E−05 |
| hsa-miR-27a-3p | BTG2 | −2.590598 | 0.000344713 |
| | DYSF | −6.910059 | 3.15265E−07 |
| | CST7 | −4.052690 | 3.37603E−05 |
| hsa-miR-12136, | STX11 | −3.499122 | 5.00642E−05 |
| hsa-miR-27a-3p | | | |
| | ABCC3 | −2.862026 | 0.005561681 |
| | IER3 | −2.335862 | 0.010406582 |
| | VNN2 | −3.533973 | 0.001575622 |
| | VNN1 | −3.397833 | 0.000498871 |
| | SOCS3 | −15.072286 | 5.32784E−12 |
| | CD163 | −3.927915 | 7.18413E−06 |
| | PIM2 | −2.244494 | 0.00061217 |
| | PPP1R15A | −2.304034 | 0.000147278 |
| | IGLV3-25 | −2.314552 | 0.04315051 |
| hsa-miR-27a-3p | ICOS | 2.153583 | 0.000494472 |
| | TLR8 | −2.562349 | 0.000284667 |
| | SAMSN1 | −3.219301 | 0.002421553 |
| | NFKBIZ | −2.611214 | 3.28634E−05 |
| hsa-miR-27a-3p | NABP1 | −2.108490 | 0.001890567 |
| hsa-miR-12136 | MCTP1 | −2.050468 | 0.002574684 |
| | EMILIN2 | −2.652938 | 4.91549E−05 |
| hsa-miR-618 | ITPRIP | −2.263424 | 0.00051204 |
| | NLRP3 | −3.013511 | 5.54862E−05 |
| | ANKRD22 | −4.432498 | 0.001936942 |
| | JAML | −2.087735 | 0.001211197 |
| | RPL10P16 | 5.442146 | 0.000138108 |
| | RPL29P4 | 3.295480 | 0.001039792 |
| | RPL10P9 | 118.708800 | 5.73245E−07 |
| | LOC441081 | −9.864287 | 3.33231E−07 |
| | NAMPTP1 | −2.863294 | 0.007482772 |
| | NCF1 | −2.204434 | 0.001291324 |
| | NCF1B | −2.042988 | 0.003796592 |
| | FCGR1CP | −5.279120 | 1.88105E−05 |
| | RNVU1-14 | 2.335143 | 0.01122683 |
| hsa-miR-12136 | LOC102723360 | −2.209455 | 0.028875165 |
| | LOC107986613 | 2.314788 | 0.012711799 |

TABLE 3

| Common | Targeted gene | Blood | |
|---|---|---|---|
| microRNA | symbol | P/C FC | raw p-value |
| hsa-miR-331-3p | AQP9 | −3.584254 | 4.2873E−05 |
| | CNN2 | −2.667414 | 0.004120064 |
| | CUX1 | −2.409842 | 0.001050514 |
| | ARID3A | −3.136860 | 6.43477E−05 |
| | PHC2 | −3.756023 | 0.00029434 |
| | GNAZ | −2.071207 | 0.023431834 |
| | LSP1 | −2.844348 | 2.88906E−05 |
| | MAFG | −3.255636 | 6.99245E−06 |
| | MNT | −2.899753 | 0.000656958 |
| | PML | −2.328198 | 0.024684715 |
| | NECTIN1 | −2.829434 | 0.001030185 |
| | SLC9A1 | −3.098512 | 0.00354239 |
| | SOD2 | −2.453118 | 0.00046603 |
| | THBS1 * | −3.114932 | 1.09916E−07 |
| | GAS7 | −3.964507 | 1.26544E−05 |
| | ADAM19 | −3.032268 | 8.91256E−05 |
| | H6PD | −2.345889 | 0.001461068 |
| | TBKBP1 | −2.860069 | 0.004664854 |
| | HDAC5 | −2.061604 | 0.004932731 |
| | RNF44 | −2.159634 | 0.026680541 |
| | PHLPP1 | −2.411466 | 0.004701039 |
| | ANKS1A | −2.027446 | 0.004719255 |
| | CDC42EP4 | −2.518654 | 0.00532939 |
| | TBC1D10B | −2.871798 | 0.001645804 |
| | MINK1 | −2.020686 | 0.028911833 |
| | C14orf132 | 3.126097 | 0.00356986 |
| | TMCC3 | −2.046601 | 0.008267345 |
| | SLC13A3 | 4.232253 | 0.005737561 |
| | CLMN | −2.035627 | 0.002072337 |
| | RAB1B | −2.323708 | 0.006393768 |
| | KLF16 | −2.545196 | 0.006686909 |
| | NACC1 | −2.391548 | 0.013145289 |
| | MBD6 | −3.183199 | 0.004316468 |
| | LOXHD1 | −3.430097 | 0.000180801 |
| | NFAM1 * | −3.754747 | 0.000448128 |
| | MSL1 | −2.244646 | 0.000582577 |
| | ZC3H12B | 2.662456 | 0.000163893 |
| | SAMD12 | 2.558944 | 0.002293958 |

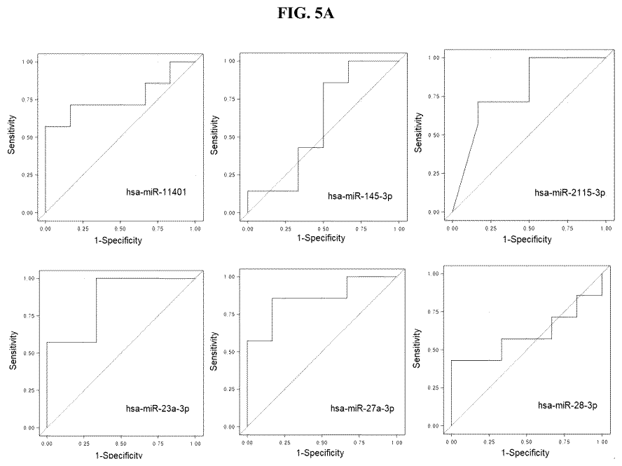

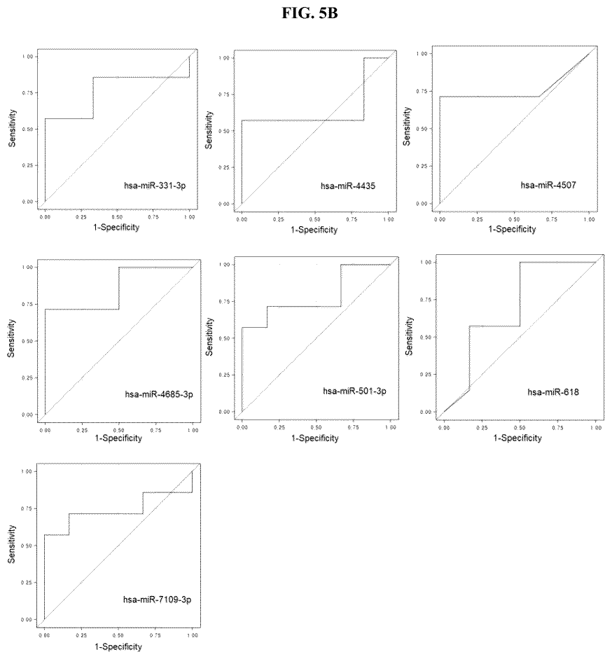

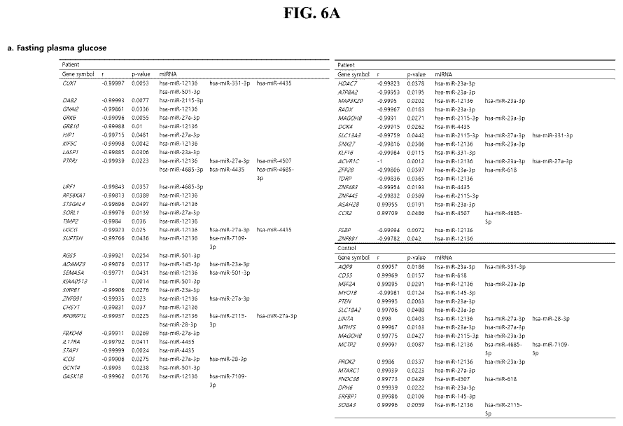

Since the miRNA signature appears uniquely for each tissue in each sample type of PD patients, the discriminative power of the miRNA signatures in the blood samples was quantified and compared to identify PD patients, and the increase or decrease in the 14 types of miRNAs of the present invention was analyzed. The results are shown in Table 4 below (FIG. 5).

TABLE 4

| | MicroRNAs in | Patient/Control | | 95% Wald | | |
|---|---|---|---|---|---|---|
| No. | blood | fold change | AUC | Confidence Limits | | p-value |
| 1 | hsa-miR-11401 | 3.077674 | 0.7619 | 0.749 | 14.132 | 0.1156 |
| 2 | hsa-miR-12136 | −5.25138 | — | — | — | — |
| 3 | hsa-miR-145-3p | −5.13289 | 0.5952 | 0.002 | 13.057 | 0.3965 |
| 4 | hsa-miR-2115-3p | −6.30624 | 0.7857 | <0.001 | 808.235 | 0.1175 |
| 5 | hsa-miR-23a-3p | −2.54386 | 0.8571 | 0.028 | 1.099 | 0.0631 |
| 6 | hsa-miR-27a-3p | −2.5836 | 0.8571 | 0.035 | 1.071 | 0.0600 |
| 7 | hsa-miR-28-3p | 3.234461 | 0.5952 | 0.720 | 2.727 | 0.3203 |
| 8 | hsa-miR-331-3p | 2.591601 | 0.7619 | 0.732 | 11.583 | 0.1293 |
| 9 | hsa-miR-4435 | 3.771794 | 0.6429 | 0.671 | 12.763 | 0.1529 |
| 10 | hsa-miR-4507 | 4.633707 | 0.7619 | <0.001 | >999.999 | 0.1692 |
| 11 | hsa-miR-4685-3p | 2.569675 | 0.8571 | 0.860 | 25.000 | 0.0744 |
| 12 | hsa-miR-501-3p | 2.798923 | 0.7857 | 0.301 | 162.756 | 0.2253 |
| 13 | hsa-miR-618 | −6.1736 | 0.7024 | 0.022 | 3.240 | 0.3004 |
| 14 | hsa-miR-7109-3p | 4.31043 | 0.7381 | 0.004 | >999.999 | 0.2022 |

### Example 5. Confirmation of Clinical Correlation of Modified Gene Expression in PD Patients

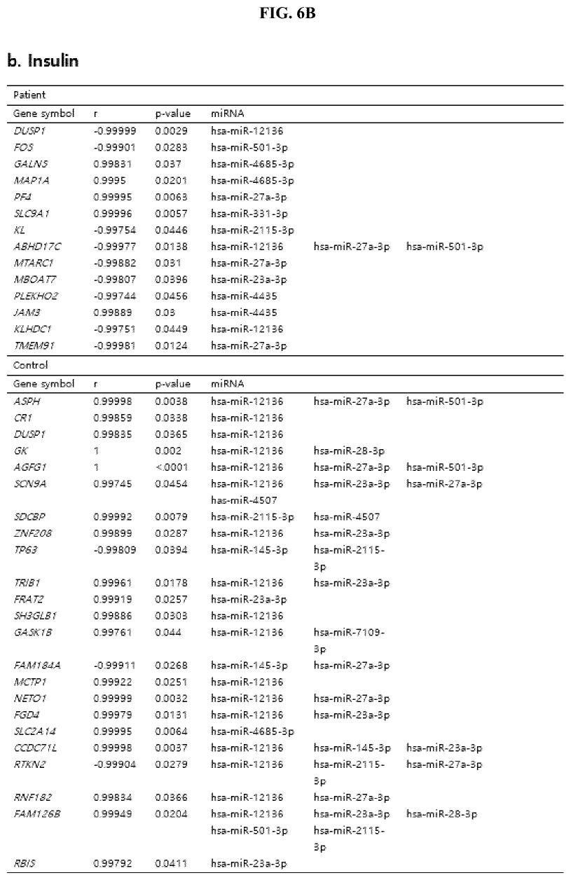

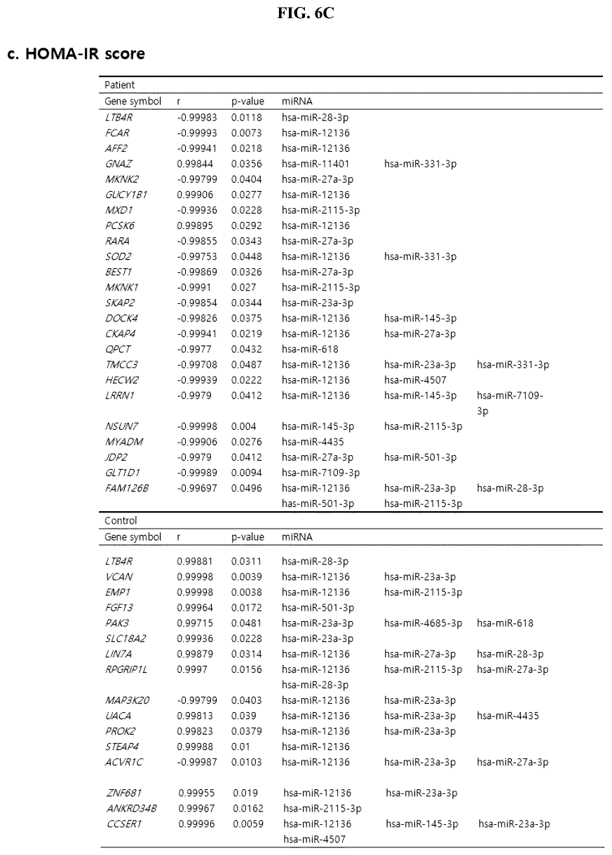

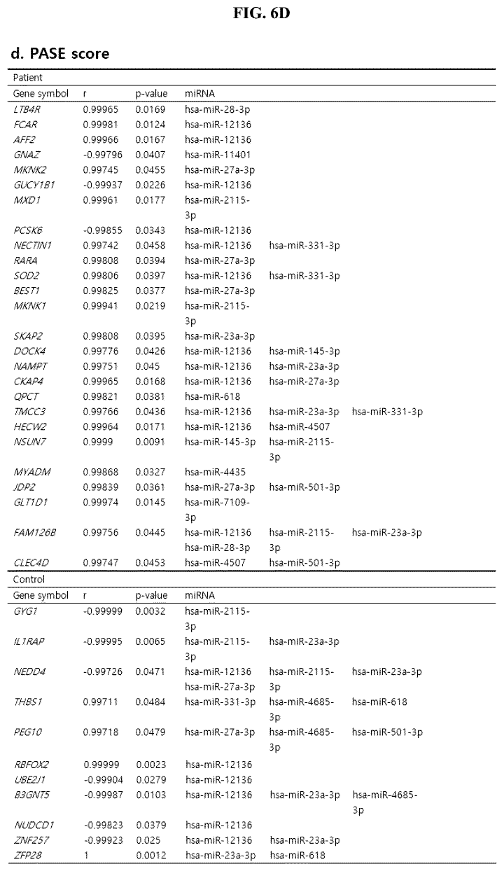

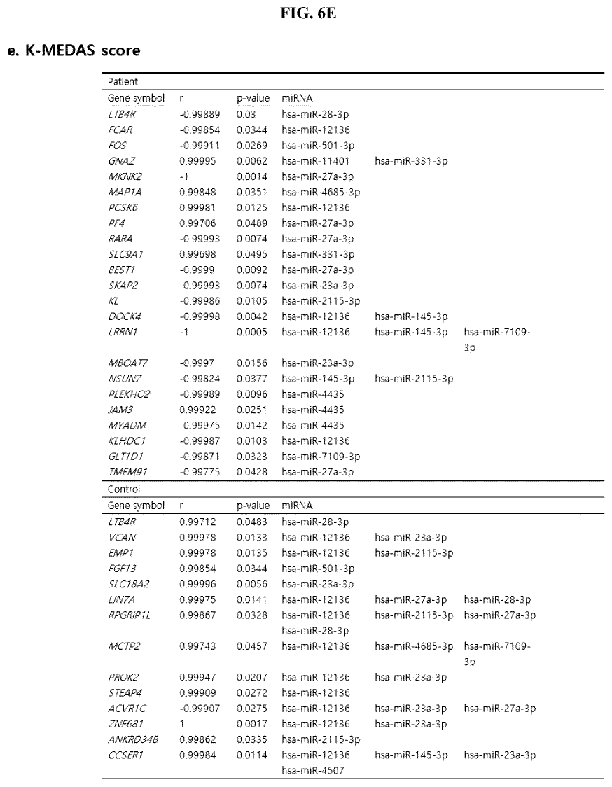

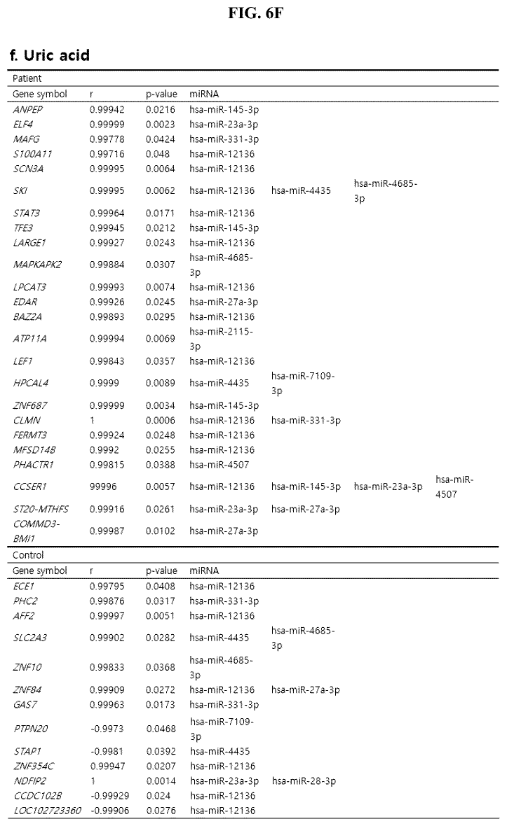

Changes in gene expression without DNA modification may occur due to environmental factors such as diet or physical activity. Therefore, the correlations of the DEGs of the PD patients with clinical factors that are known to affect the prognosis of PD were evaluated, including fasting plasma glucose, serum insulin, insulin resistance (HOMA-IR score), uric acid, dietary pattern (K-MEDAS score), and physical activity (PASE score). DEGs with a significant partial correlation coefficient (r) and p-value are shown in FIGS. 6A to 6F.

In particular, the same clinical factors affected different DEGs in the PD patients compared to the controls, and the same genes in the PD patients were influenced by different clinical factors compared to the controls. A variety of DEGs in the blood samples were correlated with these environmental factors, but the environmental influences on the gene expression were also altered in the PD patients compared to the controls in the blood samples.

### Example 6. Confirmation of the Interaction Network of Target Genes with DEmiRNAs in Blood

In the living body, two or more proteins interact to form a highly complex network and tightly controlled protein interactions underlie nearly all life phenomena. Accordingly, in the present invention, the protein-protein interaction (PPI) network was confirmed using the Search Tool for Interacting Genes (STRING) database with a confidence threshold of 0.2.

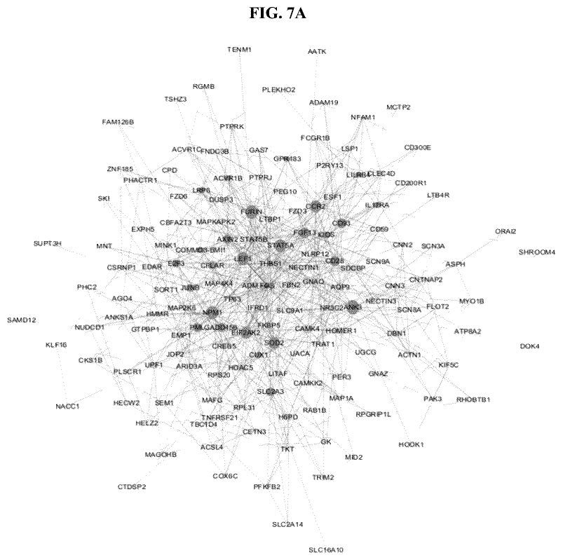

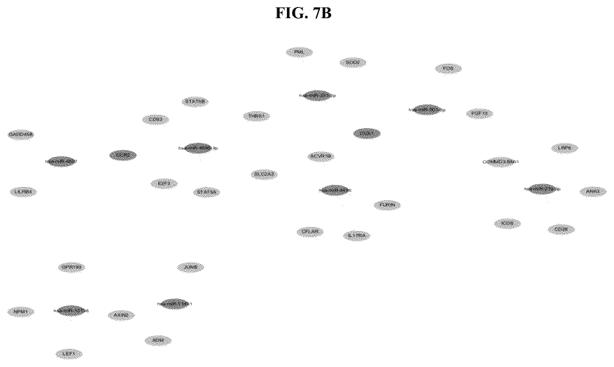

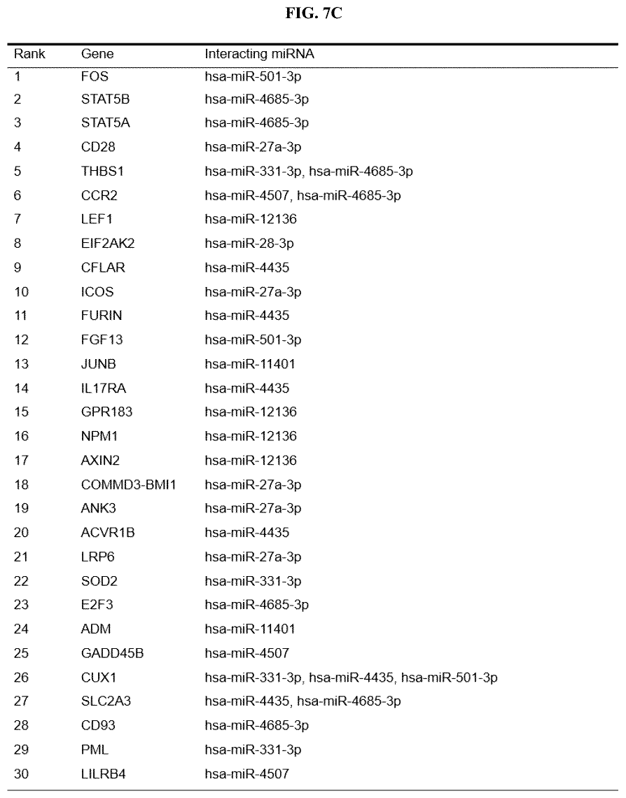

As a result. The interaction network of target genes and differentially expressed miRNAs (DEmiRNAs) identified by protein-protein interaction network analysis showed either up-down or down-up regulation in blood (FIGS. 7A and 7B). Among these, 8 DEmiRNAs interacted with the top 30 hub genes in blood (FIG. 7C).

The description of the present invention described above is for illustrative purposes, and those skilled in the art will understand that the present invention can be easily modified into other specific forms without changing the technical ideas or essential features of the present invention. Therefore, the embodiments described above should be understood as illustrative in all respects and not restrictive.

What is claimed is:

1. A method comprising the steps of:
(S1) measuring the level of any one or more of miRNAs selected from the group consisting of hsa-miR-11401, has-miR-12136, hsa-miR-145-3p, hsa-miR-2115-3p, hsa-miR-23a-3p, hsa-miR-27a-3p, hsa-miR-28-3p, hsa-miR-331-3p, hsa-miR-4435, hsa-miR-4507, hsa-miR-4685-3p, hsa-miR-501-3p, hsa-miR-618, and hsa-miR-7109-3p in a biological sample isolated from a subject;
(S2) comparing the level of the miRNA with the level of the miRNA in a biological sample isolated from a normal control group;
(S3) diagnosing a subject as having Parkinson's disease (PD), when among the miRNAs in a biological sample isolated from the subject:
(a) the level of one or more of miRNAs selected from the group consisting of hsa-miR-11401, hsa-miR-28-3p, hsa-miR-331-3p, hsa-miR-4435, hsa-miR-4507, hsa-miR-4685-3p, hsa-miR-501-3p, and hsa-miR-7109-3p is increased compared to the level of the miRNAs in a biological sample isolated from a normal control group; or
(b) the level of one or more of miRNAs selected from the group consisting of hsa-miR-12136, hsa-miR-145-3p, hsa-miR-2115-3p, hsa-miR-23a-3p, hsa-miR-27a-3p, and hsa-miR-618 is decreased compared to the level of the miRNAs in a biological sample isolated from a normal control group; and
(S4) treating the PD with chemotherapy, surgery, or biological therapy when PD is diagnosed in step (S3).

2. The method of claim 1, wherein the biological sample is one selected from the group consisting of a blood sample, a serum sample, a whole blood sample, and a plasma sample.

3. A method comprising the steps of:
(S1) measuring the level of any one or more of miRNAs selected from the group consisting of hsa-miR-11401, has-miR-12136, hsa-miR-145-3p, hsa-miR-2115-3p, hsa-miR-23a-3p, hsa-miR-27a-3p, hsa-miR-28-3p, hsa-miR-331-3p, hsa-miR-4435, hsa-miR-4507, hsa-miR-4685-3p, hsa-miR-501-3p, hsa-miR-618, and hsa-miR-7109-3p in a biological sample isolated from a subject;
(S2) comparing the level of the miRNA with the level of the miRNA in a biological sample isolated from a normal control group;
(S3) diagnosing a subject as having PD, when among the miRNAs in a biological sample isolated from the subject:
(a) the level of one or more of miRNAs selected from the group consisting of hsa-miR-11401, hsa-miR-28-3p, hsa-miR-331-3p, hsa-miR-4435, hsa-miR-4685-3p, and hsa-miR-501-3p is more than 2 fold change increase compared to the level of the miRNAs in a biological sample isolated from a normal control group, and the AUC value is ≥0.5, the p-value is <0.5, the Confidence Limits value falls between 0.001 and 165; or
(b) the level of one or more of miRNAs selected from the group consisting of hsa-miR-145-3p, hsa-miR-23a-3p, hsa-miR-27a-3p, and hsa-miR-618 is more than 2 fold change decreased compared to the level of the miRNAs in a biological sample isolated from a normal control group, and the AUC value is ≥0.5, the p-value is <0.5, the Confidence Limits value falls between 0.001 and 165; and
(S4) treating the PD with chemotherapy, surgery, or biological therapy when PD is diagnosed in step (S3).
